(19) United States
(12) Patent Application Publication
(10) Pub. No.: US 2025/0108004 A1
(43) Pub. Date: Apr. 3, 2025

(54) AEROSOL DELIVERY OF EDTA COMPOSITIONS

(71) Applicant: SterileCare Inc., Markham (CA)

(72) Inventor: Karen Bernice Mueller, Holland Landing (CA)

(21) Appl. No.: 18/896,145

(22) Filed: Sep. 25, 2024

Related U.S. Application Data
(60) Provisional application No. 63/541,693, filed on Sep. 29, 2023.

(51) Int. Cl. A61K 9/00; A61K 31/137; A61K 31/198; A61K 31/505; A61K 31/65; A61K 47/18; A61P 31/04

(52) U.S. Cl. CPC A61K 9/0078; A61K 31/137; A61K 31/198; A61K 31/505; A61K 31/65; A61K 47/183; A61P 31/04

(57) ABSTRACT

Improved systems, compositions, and methods of treating infections with antimicrobials are provided, having reduced risk of creating or promoting drug-resistant microbes or superinfections, where one or more antimicrobials susceptible to drug-resistant organisms are applied via an aerosol delivery device in combination with an EDTA solution at elevated pH.

### CROSS-REFERENCE TO RELATED APPLICATIONS

This application claims priority to U.S. Provisional Application No. 63/541,693 titled “AEROSOL DELIVERY OF EDTA COMPOSITIONS” filed Sep. 29, 2023, the entire contents of which are incorporated by reference herein.

### FIELD OF THE INVENTION

This invention pertains to products, methods of manufacture, and methods of use for delivery of ethylenediaminetetraacetic acid (EDTA) compositions in aerosol or mist form to the lungs, mucous membranes, or other surfaces of the body for medical purposes such as treating infections.

### BACKGROUND

Antibiotics have long been used to fight various infections or other ailments. However, after prolonged use, drug-resistant microbes have emerged for many once popular antibiotics such as tetracycline, penicillin, etc., making such drugs less useful. For example, streptomycin, discovered in 1943, was the first effective tool for treating tuberculosis and led to a dramatic decline in tuberculosis deaths worldwide. But as early as 1946, physicians at the Mayo Clinic observed strains of bacteria carried by tuberculosis patients that had become roughly 1,000 times as resistant to streptomycin than other strains. Today, streptomycin and related aminoglycosides are threatened with obsolescence due to rising drug resistance in microbes.

There is a need for improved treatments that can overcome the ongoing challenges of drug-resistant microbes, including reducing the risk of creating drug-resistant microbes when treating an infection or better treating infections from drug-resistant organisms, including multidrug-resistant organisms (MDROs) such as Methicillin-resistant Staphylococcus aureus (MRSA) or multidrug-resistant Mycobacterium tuberculosis (MDR-TB).

There is also a need for improved delivery of pharmaceutical systems in which synergy between a drug and other compounds may be realized, as well as a need for improved systems of treating infection that do not rely on high doses of drugs in the bloodstream. Further, there is a need to apply existing antibiotics in ways that reduce the risk of developing drug-resistant mutants of the targeted microbe and reduce the risk of superinfections from other microbes when treating a patient systemically.

A wide range of drug delivery options and treatment methods are possible given the diverse array of cell types and surface materials in the lungs, including the epithelium, endothelium, alveolar macrophages, interstitium and basement membrane, lymphatic system, epithelial lining fluid, lung surfactants, and mucociliary clearance of substances in the lungs. There is a need for improved interaction of pharmaceutical systems with one or more of these surfaces and for improved treatment of infections that may affect one or more such surfaces or materials while reducing the risk of creating new drug-resistant species or MDROs, or to reduce the risk of creating superinfections.

The many challenges faced in infections of the lungs are illustrated by the unfortunate state of treatment for cystic fibrosis patients, as explained by Daniel J. Hassett et al., “Despite the tremendous early success of CFTR modifier drug therapy [therapy useful for some mutations to directly modify mutated proteins that lead to cystic fibrosis], current evidence indicates that airway infection will continue to be a cause of morbidity and premature mortality in CF. This is especially true for the large proportion of persons with CF whose lung disease preceded the advent of CFTR modifier therapy. . . . In these individuals, particularly those with moderate to severe lung disease, chronic infection, often with MDR-PA [multiple drug-resistant Pseudomonas aeruginosa] bacteria, presents an important and serious challenge to clinical care. Unfortunately, the development of novel antibiotics to meet this challenge has faltered, with no new classes of antibiotics being developed in the last 20 years.” (Frontiers in Microbiology, 12 [2021]: 639362, doi: 10.3389/fmicb.2021.639362.)

The widespread lack of new classes of antibiotics creates a need for improved uses of existing antibiotics, even those that are declining due to the rising difficulty of drug resistance and especially multiple drug resistance, and a great need for new approaches to mitigate or contain the problems of drug resistance.

In pointing to particular problems or opportunities, unless otherwise indicated, Applicant is not suggesting that any particular aspect claimed herein must necessarily address each or any of the problems or opportunities stated, though it is believed that various aspects described herein may generally address at least one of the problems or opportunities pointed to, or may instead provide other significant benefits not necessarily specified above.

### SUMMARY

Provided herein are methods for treating an infection in a patient in need thereof, comprising providing a therapeutically effective dose of an antimicrobial susceptible to drug-resistant organisms provided in an aqueous medium comprising a therapeutically effective amount of EDTA at a pH from 8.5 to 11, and delivering the aqueous medium via an aerosol delivery device to the patient. The method has a reduced risk of creating disease-resistant organisms.

Further provided herein is an aerosol delivery system for treating a respiratory ailment in a patient in need thereof, wherein the system comprises a nebulizer having a nebulizing chamber and a pharmaceutical composition comprising a therapeutically effective amount of one or more antimicrobials and EDTA at a pH of 8.5 to 11, wherein the pharmaceutical composition is nebulized in the nebulizing chamber and delivered as an aerosol to a patient.

Further provided herein is a liquid pharmaceutical composition comprising an antimicrobial and EDTA, wherein the pharmaceutical composition has a pH of greater than 8.5.

### BRIEF DESCRIPTION OF THE DRAWINGS

FIG. 1 depicts a nebulizer apparatus capable of delivering EDTA and antibiotics for the treatment of respiratory ailments.

### DETAILED DESCRIPTION

Provided herein are systems and methods for treating an infection in a subject in need thereof while reducing the risk of creating disease-resistant organisms. The systems and methods utilize a composition that includes EDTA and a therapeutically effective amount of an antimicrobial at a pH greater than 8.5.

A pharmaceutical composition is provided for use in generating a spray or aerosol comprising a therapeutically effective concentration of an antimicrobial and from about 0.1 wt % to about 15 wt % EDTA at a pH of 8.5 or higher. The pharmaceutical composition may be a liquid solution.

The pharmaceutical composition includes EDTA or a pharmaceutically acceptable salt thereof. For example, the EDTA may be tetrasodium EDTA, disodium EDTA, sodium calcium edetate, or a combination thereof.

The EDTA may be present in the pharmaceutical composition in an amount from about 0.1% to about 15%. For example, the EDTA may be present in the pharmaceutical composition in an amount from about 0.1% to about 0.5%, about 0.1% to about 1%, about 0.1% to about 5%, about 0.1% to about 10%, about 0.1% to about 15%, about 0.5% to about 15%, about 1% to about 15%, about 5% to about 15%, about 10% to about 15%, about 1% to about 5%, about 1% to about 10%, or about 5% to about 10%. As another example, the EDTA may be present in the composition in an amount of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, or about 15%. In some embodiments, the EDTA may be present in the pharmaceutical composition in an amount from about 0.2% to about 12%, or from about 0.5% to about 11%.

The pharmaceutical composition may have a pH of about 8.5 or higher. For example, the composition may have a pH of about 8.5 or higher, about 9.0 or higher, about 9.5 or higher, about 10.0 or higher, about 10.5 or higher, about 11.0 or higher, or about 11.5 or higher. As another example, the pharmaceutical composition may have a pH from about 8.5 to about 9.0, about 8.5 to about 9.5, about 8.5 to about 10.0, about 8.5 to about 10.5, about 8.5 to about 11.0, about 8.5 to about 11.5, about 9.0 to about 11.5, about 9.5 to about 11.5, about 10.0 to about 11.5, about 10.5 to about 11.5, about 11.0 to about 11.5, about 9.0 to about 11.0, or about 9.5 to about 10.5. As another example, the composition may have a pH of about 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, or about 11.5.

In some aspects, the elevated pH for optimum effect of EDTA may also bring further benefits to the lungs, where acidification of the airways can be a serious concern, being associated with bronchospasm, inflammation (neutrophilic and eosinophilic), hyper-reactivity of the bronchia, ciliary dysfunction, abnormal fluid transport, epithelial dysfunction, oxidative damage, and alteration of cellular death pathways, including inhibition of apoptosis. Suitable buffer systems, described below, may be employed to enhance the alkaline treatment of acidified airways or to give a longer lasting benefit from the EDTA or other components.

The pharmaceutical composition may further include a physiologically acceptable concentration of sodium chloride, whether isotonic (about 0.9 wt % or from 0.7 wt % to 0.9 wt %), slightly hypotonic (e.g., from 0.6 wt % to 0.75 wt %), or hypertonic (e.g., from 0.9 wt % to 11 wt %, such as 0.9 wt %, 1.3 wt %, 1.5 wt %, 3.5 wt %, 5 wt %, 7 wt %, or 9 wt %).

The combination of EDTA with antimicrobials as described herein may not only be effective in inhibiting or destroying pathogens but, in some aspects, may be effective in mitigating harmful biofilm in the body, such as biofilm in the lungs, nasal sinuses, in the ears, etc. Without wishing to be bound by theory, it is believed that EDTA's contribution in mitigating or undermining biofilm in the methods and compositions described herein may involve its ability to sequester certain materials such as calcium, magnesium, zinc, and iron that may be needed for bacterial growth and biofilm formation. The ability of EDTA to chelate cations may help remove ions essential for the extracellular polymeric substances that biofilm-formers use to adhere to surfaces and protect a colony of microbes. Such effects may involve interfering with microbial enzymes, quorum signaling, the structure of extracellular polymeric substances, etc. Significant gains in performance may be obtained by combining EDTA, particularly in its tetrasodium form at elevated pH.

It is further believed that in at least some aspects, a reduced tendency to promote drug resistance or a reduced risk for superinfection or creation of MRDOs can be achieved by reduction of systemic application of the antibiotics by instead targeting their application to zones of infections such as the lungs or other specific zones of infection. The use of sprays or aerosols may be of particular usefulness and may be used in some aspects to deliver a pharmaceutically effective dose locally without giving a systemic dose capable of promoting drug resistance or superinfection.

The antimicrobial combined with EDTA and applied via aerosol or spray may be relatively poorly absorbed by contact with mucous membranes, lung tissue, or the gastrointestinal tract, such that the application of a therapeutically effective dose to treat a localized infection such as an infection in the lungs, ears, nose, or throat can be achieved without substantial amounts of the drug entering the bloodstream. In this way, the drug concentration as applied to the lungs or other membranes can be therapeutically effective while in the presence of EDTA at elevated pH, thereby maintaining conditions for synergy with the antimicrobial and achieving positive results, at least in part through a local rather than systemic effect. In related aspects, the antimicrobial may be polar, lipophilic, both polar and lipophilic, or may have a molecular weight of 450 or greater, 550 or greater, 650 or greater, 750 or greater, 1,000 or greater, 1,500 or greater, or 2,000 or greater.

The composition may further include lipids to prepare liposomes. This may be accomplished with freeze-drying, spray drying, sonication, reverse phase evaporation, and other methods known in the art. The liposomes may be dispersed in liquid and delivered by a nebulizer. Nanoemulsions may also be used. Ectoins may be useful as part of the emulsion system for liposomes or nanoemulsions.

Thus, in some aspects, after delivering an effective amount of the drug via aerosol or spray to a local region of the body, any subsequent absorption into the bloodstream is minimal or undetectable.

In one aspect, a convenient and useful system and method is provided in which a solution comprising EDTA at a pH greater than 8.5, such as from 9 to 11, is provided in combination with a pharmaceutically effective amount of an antibiotic, wherein the antibiotic is known to be challenged by drug-resistant microbes, or known to promote the rise of drug-resistant microbes when used conventionally, and wherein synergy between the EDTA and the antibiotic assists in treating an infection. In some aspects, the synergy may be realized through a suitable delivery mechanism other than systemic application in the bloodstream but through localized delivery via aerosol to the lungs or affected mucous membranes.

The antimicrobial may be included in the pharmaceutical composition in an amount from about 0.01 wt % to about 20 wt %, such as from about 0.1 wt % to about 10 wt %, or from about 0.1 wt % to about 3 wt %. For example, the antimicrobial may be included in the pharmaceutical composition an amount from about 0.01 wt % to about 0.1 wt %, about 0.01 wt % to about 1 wt %, about 0.01 wt % to about 5 wt %, about 0.01 wt % to about 10 wt %, about 0.01 wt % to about 20 wt %, about 0.1 wt % to about 20 wt %, about 1 wt % to about 20 wt %, about 5 wt % to about 20 wt %, about 10 wt % to about 20 wt %, about 0.1 wt % to about 5 wt %, or about 0.1 wt % to about 10 wt %. Those having skill in the art will be capable of determining the appropriate therapeutic dose of the antimicrobial to administer to the patient in need thereof.

The antimicrobial may include one or more antibiotics. In particular, the antibiotic may include an antibiotic susceptible to drug-resistant organisms. Such antibiotics may include those from major drug classes such as:
Aminoglycosides, such as amikacin, gentamicin, gramicidin, neomycin, streptomycin, and tobramycin;
Avermectins, such as ivermectin;
Carbapenems, such as doripenem, ertapenem, imipenem, and meropenem;
Cephalosporins, such as cefazolin, cefepime, cefotaxime, ceftriaxone, cephalosporins, and cephalexin;
Cyclic peptides, such as bacitracin, cyclosporine, and ramoplanin;
Dihydrofolate reductase (DHFR) inhibitors, such as trimethoprim, pyrimethamine proguanil, brodimoprim, and iclaprim;
Echinocandins, such as caspofungin, micafungin, and anidulafungin;
Fluoroquinolones, such as ciprofloxacin, levofloxacin, and moxifloxacin; Fusidanes, such as fusidic acid and sodium fusidate;
Glycopeptides, such as teicoplanin and vancomycin;
Imidazole antifungals, such as clotrimazole, econazole, ketoconazole, miconazole, and tioconazole;
Lincosamides, such as clindamycin;
Lipopeptides and linear peptides, such as daptomycin, iturin, nisin, polymyxin B, colistin (polymyxin E), surfactin, and tyrothricin;
Macrolides, such as azithromycin, clarithromycin, and erythromycin;
Monocarboxylic acids, such as mupirocin;
Monobactams, such as aztreonam;
Nitrofurans, such as nitrofurazone, furazolidone, furaltadone, and nitrofurantoin;
Nitroimidazoles, such as metronidazole, tinidazole, nimorazole, dimetridazole, pretomanid, ornidazole, megazol, azanidazole, and benznidazole;
Oxazolidinones, such as linezolid, contezolid, sutezolid, eperezolid, radezolid, posizolid, tedizolid, and delpazolid;
Penicillins, such as amoxicillin, ampicillin, methicillin, and penicillin;
Phenicols, such as chloramphenicol, azidamfenicol, florfenicol, and thiamphenicol; Pleuromutilins, such as tiamulin, valnemulin, and retapamulin;
Polyene antifungals, such as nystatin, natamycin/pimaricin, hamycin, and amphotericin B;
Rifamycins, such as rifampin, rifabutin, rifapentine, and rifalazil;
Sulfonamides, such as sulfamethoxazole, sulfonamides, sulfadiazine, silver sulfadiazine, sulfacetamide, sulfasalazine, sulfathiazole, sulfisoxazole, and sulfadoxine;
Sulfones, such as dapsone, sulfanilamide, and sulfapyridine;
Tetracyclines, such as tetracycline, doxycycline, minocycline, tigecycline, eravacycline, and omadacycline;
Triazoles, such as fluconazole, itraconazole, voriconazole, posaconazole, and isavuconazole;
Tuberactinomycin, such as viomycin, enviomycin, and capreomycin; and Others, such as quinupristin and dalfopristin, chlorhexidine, peroxides, silver nanoparticles; and combinations of any of the antibiotics or classes of antibiotics listed above.

The antimicrobial may include one or more antifungal agents. In some aspects, EDTA coupled with antifungal agents can effectively improve efficacy and reduce the risk of new drug resistant organisms or mitigate drug resistance in targeted or peripheral organisms. The antifungal agent may include amphotericin, nystatin, pimaricin, fluconazole, itraconazole, amorolfine, ketoconazole, naftifine, terbinafine, 5-fluorocytosine, or any combination thereof.

The pharmaceutical composition may include a mucolytic agent. Mucolytic agents may reduce the viscosity of mucous by reducing S—S bonds to S—H (sulfhydryl) bonds, thereby disrupting the crosslinking that gives mucous its body. The mucolytic agent may be combined with the EDTA and the antimicrobial in a single composition. The mucolytic agent may comprise-acetyl cysteine, bromhexine, carbocysteine, erdosteine, fudosteine, peptide mucolytics, dornase alfa, hypertonic saline solution, or another mucolytic agent known in the art or a combination thereof. In particular, carbocysteine has an expectorative effect and, in some cases, can reduce bacterial load in the airways. Furthermore, erdosteine and fudosteine are mucolytic thiol derivatives and may have additional anti-oxidant and anti-tussive actions. Peptide mucolytics tend to preserve the protective mucins but target DNA polymers and F-actin links and can be useful in dealing with cystic fibrosis. Dornase alfa functions by depolymerizing DNA polymers. Thymosin β4 interacts with F-actin to yield its mucolytic effect. Bromhexine can disrupt the structure of mucopolysaccharides and glycoproteins and break sulfur bonds within mucoproteins, or assist in other ways.

The mucolytic agent may be present in the pharmaceutical composition in an amount from about 5 wt % to about 20 wt %. For example, the mucolytic agent may be present int eh pharmaceutical composition in an amount from about 5 wt % to about 10 wt %, about 5 wt % to about 15 wt %, about 5 wt % to about 20 wt %, about 10 wt % to about 20 wt %, or about 15 wt % to about 20 wt %. In other examples, the mucolytic agent may be present in an amount of about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or about 20 wt %.

The compositions, treatments, and methods described herein may be applied when suitable to humans of any age, but may also be useful in treating non-human mammals or other animals.

In addition to potential synergy between EDTA solutions and an antibiotic, in some aspects, further synergy may also be realized with the presence of one or more additional ingredients that are compatible with the conditions required for the EDTA-drug combination while also being effective in providing further synergy by, for example, weakening the protective material of a biofilm, interfering with microbial enzymes, or other effects that can enhance the efficacy of the drug-EDTA combination.

Such additional active agents may include biofilm mitigation agents such as N-acetyl cysteine, cysteine such as D-cysteine or cysteine salts (sodium, potassium, etc.), O-Acetylserine (OAS), glutathione, catechins such as EGCG, ectoin, panthenol or pantothenic acid or derivatives thereof, nitric oxide (NO)-generating compounds, or enzymes such as DNAse, lysozymes, proteinase, etc., or any combination thereof. For example, without wishing to be bound by theory, it is believed that one mechanism by which N-acetyl cysteine can undermine biofilms is through disrupting sulfide bonds. This mechanism may have synergy with EDTA's proposed mechanisms of biofilm mitigation through chelation of certain cations. It is proposed that both may also inhibit enzymes associated with the virulence of biofilms and biofilm formation by altering the enzymes by attacking sulfide bonds and interfering with cations that are essential for some such enzymes. Thus, in one aspect, a composition and method for treating infection sites where biofilm is a threat may include a combination of EDTA, N-acetyl cysteine, and one or more antibiotics, wherein the application of the EDTA and N-acetyl cysteine (or EDTA alone) may weaken a biofilm and thereby enhance the ability of the antibiotic to control the targeted microbes.

The pharmaceutical composition may include an enzyme inhibitor. Enzyme inhibitors may be provided to undermine enzymes from various microbes that may contribute to drug resistance. Stated another way, the enzyme inhibitor may be adapted to inhibit enzymes known to assist drug resistance relative to an antimicrobial included in the pharmaceutical composition.

In some aspects, ectoin may be applied in the lungs to reduce inflammation of airways and assist in healing while also serving as an effective antimicrobial against some bacteria such as Pseudomonas aeruginosa and Staphylococcus aureus.

The pharmaceutical composition may include NO-generating compounds to enhance lung performance and/or enhance antimicrobial activity. Such NO-generating compounds may include, for example, NONOates (also described as NONO-ates) in which the NONOate functional group —O—N═N—O—, a stabilized dimer of nitric oxide, can release NO over time in physiological settings or serve as a nitric oxide donor. Many parent amines can be used to prepare NONOates, giving rise to a variety of compounds such as spermine NONOate, DETA NONOate, PROLI NONOate, and EDTA NONOate. The pharmaceutical composition may include spermine NONOate, DETA NONOate, PROLI NONOate, EDTA NONOate, the drug AB569, sodium nitrite or other acidified nitrites, polyamidoamines (including hyperbranched polyamidoamines), polykanamycins (including hyperbranched polykanamycins), sodium nitroprusside, S-nitrosoglutathione, NO-releasing alginates, chitosan oligosaccharides, cephalosporin-based NO donor prodrugs (including cephalosporin-3′-diazeniumdiolates and diethylamin-cephalosporin-3′-diazeniumdiolate), or a combination thereof. In particular, and without wishing to be bound by theory, the combination of EDTA with EDTA NONOate is believed to be relatively stable in the pH range where EDTA is particularly effective as an antimicrobial, such as from pH 9 to 11, and may provide particular benefits in fighting microbial infections and/or in controlling biofilm. In some embodiments, the NO-generating compound may be combined with N-acetyl cysteine, panthenol, oligoethylene glycol, hydrophobic ethylhexyl, or cationic primary amines.

The NO-generating compound may be present in the pharmaceutical composition in an amount from about 1 wt % to about 20 wt %. The pharmaceutical composition may include a pharmaceutically effective buffer. The buffer may include an alkaline glycine buffer (e.g., a glycine solution adjusted with NaOH to a pH of 10 or 10.5), a Tris buffer (tris(hydroxymethyl)aminomethane); a carbonate-bicarbonate buffer with sodium carbonate and sodium bicarbonate; or a HEPES buffer (4-(2-hydroxyethyl)-1-piperazineethanesulfonic acid).

The buffer may be present in the pharmaceutical composition in an amount from about 5 wt % to about 60 wt %. For example, the buffer may be present in the pharmaceutical composition in an amount from about 5 wt % to about 10 wt %, about 5 wt % to about 20 wt %, about 5 wt % to about 30 wt %, about 5 wt % to about 40 wt %, about 5 wt % to about 50 wt %, about 5 wt % to about 60 wt %, about 10 wt % to about 60 wt %, about 20 wt % to about 60 wt %, about 30 wt % to about 60 wt %, about 40 wt % to about 60 wt %, or about 50 wt % to about 60 wt %. In other examples, the buffer may be present in the pharmaceutical composition in an amount of about 5 wt %, 10 wt %, 15 wt %, 20 wt %, 25 wt %, 30 wt %, 35 wt %, 40 wt %, 45 wt %, 50 wt %, 55 wt %, or about 60 wt %.

The pharmaceutical composition may include a solubilizer or surfactant. The solubilizer or surfactant may be present in the pharmaceutical composition in an amount from about 0.1 wt % to about 20 wt %, such as from about 0.5 wt % to about 10 wt %. The solubilizer or surfactant may include Kolliphor EL (a nonionic solubilizer and emulsifier made by reacting castor oil with ethylene oxide), glycerin, isopropylidene glycerol, 1,2 propanediol, 1,3 propanediol, propylene glycol, glycerol, non-toxic reaction products of various lipids with ethylene oxide, or other solubilizers and surfactants known in the art. In particular, these solubilizers and surfactants may help stabilize proteins in the pharmaceutical composition.

The pharmaceutical composition may include a salt, such as sodium chloride, potassium chloride, sodium citrate, a carbonate salt, a phosphate salt, a bicarbonate salt, or other pharmaceutically acceptable salts. The salt may be present in the pharmaceutical composition in an amount of up to about 2 wt %, such as up to about 1 wt %. In particular, sodium chloride may be included in an amount from about 0.4 wt % to about 1.5 wt %.

The pharmaceutical composition may include an amino acid. Certain amino acid groups assist in protein or peptide solubility. Aspartic acid, glutamic acid, and serine in particular have been noted for strong contributions to solubility. When antibiotic peptides and other drugs are relatively low in solubility-enhancing amino acids, achieving a nebulizable solution may require the addition of excipients (e.g., solvents, surfactants, stabilizers, etc.) that assist in solubilizing peptides, enhancing their suspension in aqueous solutions, or assist in their delivery via nebulization.

The pharmaceutical composition may further include other additives or active ingredients useful in treating respiratory infections, including peptides suitable for asthma relief, bronchodilators, cough suppressants, or a combination thereof.

In some embodiments, the pharmaceutical composition may be substantially free of peptides having a molecular weight of less than 40,000, such as less than 20,000, less than 10,000, or less than 5,000.

Combinations of EDTA and active pharmaceutical ingredients and novel methods of application may be used to treat infections in the lungs, the sinuses, the mouth or throat, or other mucous membranes in a subject in need thereof. Both the active pharmaceutical ingredient and the EDTA may be delivered via an aerosol delivery device such as an inhaler, a nebulizer or atomizer, a nasal spray device, etc., wherein a substantial fraction (e.g., at least 25%, 40%, or 60%) of the delivered liquid may have a particle size under 10 microns (e.g., for nasal sprays), 8 microns, 6 microns (e.g., for nebulizers), 5 microns, 3 microns, or 2 microns. In some aspects, a prepackaged nebulizer system is provided in which a therapeutically effective dose of the antimicrobial is provided in an ampule, cartridge, or other packaging unit that can be easily connected to the nebulizer for delivery. Any suitable kind of nebulizer, such as jet, ultrasonic, mesh, and venturi nebulizers, may be targeted to specific parts of the lungs when needed and/or may be breath-synchronized.

In some aspects, the aerosol is formed from a single fluid or suspension that is aerosolized. In other aspects, two or more steams of aerosol droplets are combined such that the ingredients in the two or more steams are kept separate until the time of delivery to the patient, which may be useful in ensuring product stability or for other purposes.

The aerosol delivery device may be disposable or reusable. Reusable nebulizers, with proper valving, may reduce medicine waste and may be more efficient. Exemplary nebulizers include breath-enhanced jet nebulizers with micro pump delivery. Nebulizers may be aided by air compressors, pumps, electric motors, etc. Nebulizer therapy may be done once or more daily as needed for a predetermined time, such as from 10 to 15 minutes or until a given dose or number of doses has been applied.

In some aspects, the aerosol delivery device may have a total output rate of over 400 mg/min, with a mass median diameter of under 5 microns, such as from 3 to 4.5 microns. The percentage respirable fraction may be over 40%, such as from 50% to 80% or from 60% to 80%.

In one aspect, one or more solid materials, such as the antimicrobial or other agents or excipients, may be delivered through the aerosol delivery device wherein the one or more solid materials are provided in a granulated state before use but are prepared for inhalation by combination with an inhalant liquid shortly before use. In this manner, a predetermined dose may be provided in an ampule or other container but kept substantially dry prior to use.

In one aspect, the aerosol delivery device is used to deliver a respiratory medicament comprising EDTA at an elevated pH and one or more antimicrobials in a flowable medicament such as an aqueous solution, suspension, slurry, colloid, etc., wherein the aerosol delivery device monitors the delivery of the medicament while also monitoring the breathing behavior of the user, and in response to the breathing behavior of the user, may adjust one or more variables associated with the aerosol delivery device and the associated treatment, including 1) altering the degree of atomization or particle size of the medicament to increase the amount of medication per unit volume of air inhaled, 2) altering the moisture content of the delivered air, 3) altering the temperature of the delivered air, 4) adjusting the air resistance, 5) providing audible, visible, or tactilely perceptible instructions to the user to modify a behavior, a setting, or take other actions, 6) adjusting the concentration of a component in the medicament.

An aerosol delivery device may be in the form of a nebulizer with a vibrating mesh. It may have a main body with a nebulizer outlet and an air channel in communication with it. The main body supports a medicine reservoir and a mesh that engages the medicine reservoir and air channel and vibrates to atomize medicine from the medicine reservoir into the air channel for discharge through the nebulizer outlet. The nebulizer outlet and mesh may be received within the patient's oral cavity when the nebulizer is in use. It may employ attaching a removable cartridge having a medicament chamber containing a powder, such as granulated antibiotics, EDTA, and excipients, which can be opened or unsealed to be combined with a liquid in a liquid chamber in the cartridge or with liquid already provided in the nebulizer. In either case, unsealing the medicament allows it to mix with a suitable liquid to form a solution or suspension, which can then be nebulized and delivered to a user.

The aerosol delivery device may employ ultrasound, a vibrating mesh, a jet, etc., to form a plume of liquid droplets having a particle diameter between one and six microns, directing the plume of particles to a mouth of a patient during an inhalation by a patient; and stopping the plume of particles during the inhalation by the patient such that a substantial majority, or nearly all, of the plume of particles, are inhaled by the patient.

In addition to those described above, a variety of other aerosol delivery device components and materials may be adapted for the purposes described herein, including those that generally operate by passing liquid through an orifice of an ultrasound instrument tip or devices having two or more nebulizing elements that may be used to deliver two or more fluids to the patient or may be used for enhanced fluid flow of a single fluid. The nebulizing elements may be vibrating elements with holes through which the fluid is expelled when the vibrating elements are vibrated.

In some aspects, the pharmaceutical composition may be driven into a nebulizer chamber using a pump such as an infusion or syringe pump. Vortical flow may also be used to create the aerosol.

In some aspects, a pre-packaged combination of solid active ingredients (e.g., EDTA and an antimicrobial) may be provided with water or a saline solution with other optional excipients, that can be brought into fluid communication with the aerosol delivery device, such as by plugging a cartridge filled with liquid and the actives into the aerosol delivery device, wherein the cartridge can be opened before, during, or after insertion to allow the liquid and the actives to flow into a chamber where it can be steadily aerosolized (nebulized) into a stream of droplets that can be carried by gas into the lungs or nose or mouth of the user. In another route, the active pharmaceutical ingredients and EDTA may be initially sealed in powder form in a chamber with optional excipients or other ingredients also in powder form, and the chamber is opened to allow the ingredients to come into contact with a liquid phase. The dry solids may be in a first chamber in a cartridge, and the separate liquid may be in a second chamber in the cartridge, with a divider between the two that can be opened (e.g., pierced or removed) to create a solution or suspension of materials that can then be aerosolized. Alternatively, the actives may enter a nebulizing chamber, mixed with a liquid that either was provided from a different source or released directly from a cartridge into the nebulizing chamber, etc.

In some aspects, the active ingredients in powder form may be delivered into a portion of a nebulizer or another form of an aerosol delivery device where a liquid is present, moved by a plunger or other displacement mechanism that pushes the powder out of a dry chamber to come into contact with the liquid. A plastic or other frangible seal may be ruptured by pressure from the plunger or prior to depressing the plunger. Capsules or ampules containing powder may also be opened by other means, such as the action of a piercing element like a blade or pin that ruptures a sealed capsule.

Portable inhalers, such as those used for delivering bronchodilators, may be used. These may, for example, have an insertable container containing the pharmaceutical composition and a housing for receiving the container.

The aerosol delivery device may have separate chambers for two or more components, such as a solid powder on one and a liquid in another, after mixing the powder and the liquid, the aerosol delivery device can be activated by pressurized gas that drives the flowable medicament (e.g., a suspension of particles or liposomes, a slurry, colloid, or solution) to leave the container under sufficient shear to form small droplets to be carried to the user's lungs, sinuses, or throat, for example. Other means may be used to rupture a powder capsule, including the use of an external magnet to pull an internal magnet through a protective membrane, thus rupturing the membrane and releasing its solid contents.

Nebulizers may provide aerosol flow directly to the user via tubing and adapters that can be placed directly into the mouth or may be provided into a covering for the mouth and nose, such as a face mask or breathing cup.

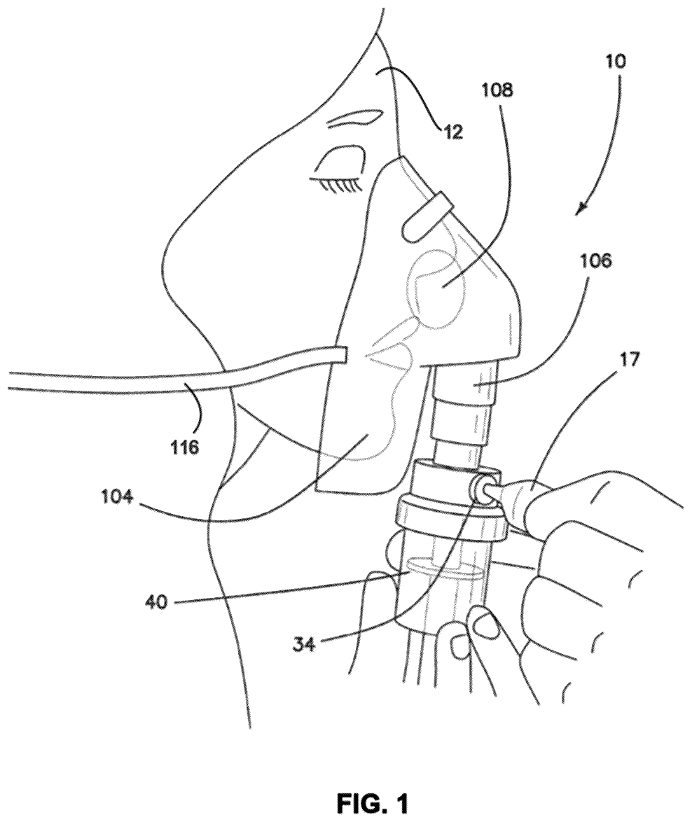

One example of an aerosol delivery device 10 is depicted in FIG. 1. Here the aerosol delivery device 10 is depicted as a nebulizer having a medication reservoir 40 (which can also serve as a nebulizing chamber) with an injection port 34 disposed directly in the reservoir 40 which may be accessed by a drug dispenser 17, adapted to deliver a dose of active ingredients in the form of a powder, slurry, suspension, colloid, or solution to enter into the liquid in the reservoir 40, thus providing direct communication between the drug dispenser 17 and the reservoir 40 via the injection port 34. The reservoir 40 is also directly coupled to a face mask 104 via a mask connector 106 disposed substantially beneath the nasal portion of the face mask 104, though it could be disposed in other sections of the face mask 104 if desired. The face mask 104 also comprises a plurality of exhaust ports 108 and can be held on the face by one or more bands 116. To use the nebulizer apparatus 10, the face mask 104 is placed over the nose and mouth of a patient 12 with the reservoir 40 already coupled to the mask connector 106 of the face mask 104. The medicated aerosol, driven by an ultrasonic mesh (not shown), an ultrasonic horn (not shown), jet or pressurized gas flow (not shown), or other means, flows from the medication reservoir 40 through the hollow mask connector 106 and to the face mask 104 of the aerosol delivery device 100. Upon inhalation or exhalation of the patient 12, the medicated aerosol flowing to the face mask 104 is then delivered to the patient 12 through their nose or mouth. The exhalation of the patient 12 is removed from the mask 104 by flowing through the exhaust ports 108. The medication reservoir 40 may be coupled to the mask connector 106 via a wide variety of connection configurations depending on the needs of the patient 12.

A method is also provided for treating an infection in a patient at reduced risk of creating disease-resistant organisms, including providing a therapeutically effective dose of a pharmaceutical composition comprising an active pharmaceutical ingredient susceptible to drug-resistant organisms provided in an aqueous medium comprising an effective amount of EDTA at a pH from 8.5 to 11, and delivering the pharmaceutical composition via an aerosol delivery device to the patient. The application of the pharmaceutical composition effectively reduces infection of the lungs, sinuses, mouth, throat, or a combination thereof.

The pharmaceutical composition may include an antibiotic susceptible to drug-resistant organisms as described herein. The antibiotic may be selected from the group consisting of amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, voriconazole, and any combination thereof.

The infection may be a respiratory infection. The respiratory infection may be a bacterial infection or a fungal infection. In some embodiments, infection may include cystic fibrosis, tuberculosis, pneumonia, or bronchitis.

The aqueous medium may further comprises at least one of the group consisting of an NO-generating compound, N-acetyl cysteine, bromhexine, ectoin, and an enzyme inhibitor, or a combination thereof. In an example, the enzyme inhibitor may be adapted to inhibit enzymes known to assist drug resistance relative to an antibiotic included in the aqueous medium. In some aspects, exhaled breath condensate (EBC) may be collected and its pH measured to assess the pH of the airway lining fluid (ALF) as a window to the state of the lungs. Since the ALF has limited buffer capacity, departures from the normal pH range of about 7.5 to 8.2 can indicate the presence of volatile acids from pathogenic causes, for example. For example, patients suffering from COPD, asthma, bronchiectasis, cystic fibrosis, and chronic cough may also have significant acidification of the airways. Providing therapeutic alkaline compositions as described herein, including the use of buffer systems to better maintain an elevated pH, may help counter some of the adverse effects of acidification in addition to good efficacy in fighting infection or other problems.

Generally, the pharmaceutical composition may be made by combining the EDTA, the antimicrobial, and optionally any of the other ingredients described herein in a carrier, such as an aqueous carrier, prior to aerosolization.

### Prophetic Examples

In a first prophetic example, a patient is diagnosed with a lower respiratory infection from gram-negative aerobic pathogens, including Pseudomonas aeruginosa, for which aztreonam is deemed suitable. To reduce the risk of superinfection from gram-positive pathogens, for which aztreonam has little effect, the antibiotic is prepared in solution with EDTA buffered with pharmaceutically acceptable agents such as alkaline glycine to give a pH of 10.5. The EDTA concentration is from 3% to 6%, and the aztreonam concentration is at 70% above the MIC for Pseudomonas aeruginosa. The antibiotic will be further diluted in the lung liquids present there. Treatment is provided in short intervals, coupled with periodic monitoring of the aztreonam concentration in the blood to ensure that the bloodstream concentration of the drug does not rise above 10% of the MIC. After several treatments, NO-generating agents are also applied to the lungs.

Aztreonam may be considered for pneumonia and, bronchitis, etc. A risk with conventional use of this drug is the production of hypervirulence in bacteria such as Pseudomonas aeruginosa. Combination with EDTA, as described in this hypothetical example, may be useful in reducing such hypervirulence or risk of superinfection and, by keeping bloodstream levels low, may also reduce the risk of promoting drug-resistant organisms in the body.

In a second prophetic example, daptomycin is used for lung infections such as pneumonia from a specific Gram-positive bacterium. It is provided in a nebulizer liquid at a concentration of 100% above the MIC with EDTA at a concentration of 2% in a hypertonic saline solution at 3% concentration and a pH of 9.3. A carbonate-bicarbonate buffer system is used. 0.3% polysorbate 80 is added as a surfactant. An ultrasonic mesh nebulizer is used to deliver the medicament.

In a third prophetic example, tetracycline is provided to a bronchitis patient with COVID. The tetracycline is in solution with 1% EDTA at a pH of 8.7 and is applied via a portable pressurized inhalant canister or via a nose spray delivered manually.

In a fourth prophetic example, a cystic fibrosis patient is treated with an aerosol delivery device adapted to deliver a 50:50 blend of tobramycin and streptomycin in a 3% EDTA solution also having 0.5% N-acetyl cysteine at a pH of 8.9 further comprising 0.2% EDTA-NONOate with an alkaline glycine/citrate buffer.

### Examples

Strains of E. coli and S. epidermidis were contacted with compositions that included tetrasodium EDTA (T-EDTA) and tetracycline. Microbroth dilution in 96-well microtiter plates was performed on the E. coli and S. epidermidis strains. 200 μL of two-fold dilutions of tetrasodium EDTA (or tetracycline) were prepared in Mueller-Hinton or Mueller-Hinton II broth. The plate contained decreasing concentrations of tetrasodium EDTA (4%-0.03%) or tetracycline (512-0.5 μg/mL) in columns 1-11. Then, 10 μL of standardized cell suspension was added to each well. The microtiter plates were incubated at 37° C. for 24 h and the results were analyzed. Each test was performed in duplicate. The MIC was determined as the lowest concentration where no growth was observed, but the adjacent wells had growth as determined by optical density. The MIC values of T-EDTA and tetracycline were determined in isolation and are provided in Table 1 below.

TABLE 1
MIC in Isolation

| | MIC in Isolation | |
|---|---|---|
| Strains | T-EDTA | Tetracycline |
| | (wt %) | (μg/mL) |
| E. Coli HC1-R2A-1 | 1 | 128 |
| S. epidermidis HC3-CA-1 | 0.0625 | 8 |

At the end of the MIC experiment, 5 μL aliquots were removed from each well in the 96-well MIC plates and used to inoculate another microtiter plate that was filled with Mueller-Hinton media. This new microtiter plate was incubated at 37° C. and growth was quantified after 24 h by measuring the optical density in the wells. The MBC was determined to be the lowest concentration of t-EDTA or Tetracycline where no growth was observed. The MBC values of T-EDTA and tetracycline were determined in isolation and are provided in Table 2.

TABLE 2
MBC in Isolation

| | MBC in Isolation | |
|---|---|---|
| | T-EDTA | Tetracycline |
| Strains | (wt %) | (μg/mL) |
| E. Coli HC1-R2A-1 | 1 | 512 |
| S. epidermidis HC3-CA-1 | 4 | 8 |

Next, compositions were prepared that included a combination of T-EDTA and tetracycline. The interaction of tetrasodium EDTA with tetracycline was investigated by the checkerboard titration method using microbroth dilution in 96-well microtiter plates. 200 μL of two-fold dilutions of tetrasodium EDTA and tetracycline were prepared in MH or MH II broth. The plate contained decreasing concentrations of tetracycline (256 μg/mL-0.5 μg/mL) in columns 1-10 and decreasing concentrations of tetrasodium EDTA (2%-0.015%) in rows A-H. Then, 10 μL of standardized cell suspension was added to each well. Microtiter plates were incubated at 37° C. for 24 h and the results were analyzed. Each test included a growth control without addition of any antimicrobials, as well as an uninoculated control column to ensure there was no contamination in the plate. The combination MIC was taken as the concentration where no growth was observed by optical density but the wells immediately adjacent had growth. To determine the MBC, 5 μL aliquots were removed from each well in the 96-well MIC plates above and used to inoculate a new microtiter plate that was filled with Mueller-Hinton media. This new microtiter plate was incubated at 37° C. for 24 h and growth was quantified by measuring the optical density in the wells. The MBC was determined to be the lowest combination of t-EDTA and Tetracycline where no growth was observed, but the wells immediately adjacent had growth. The MIC and MBC were once again determined with the combinations, and the results are shown in Tables 3 and 4, respectively.

The potential interaction of the two antimicrobials was determined by the FICI (fractional inhibitory concentration index) and FBCI (fractional bactericidal concentration index). Note: tetracycline=tet.

$$FICI = \frac{MIC of tet . in combiantion with EDTA}{MIC of tet . alone} + \frac{MIC of EDTA in combination with tet .}{MIC of EDTA alone}$$

$$FBCI = \frac{MBC of tet . in combiantion with EDTA}{MBC of tet . alone} + \frac{MBC of EDTA in combination with tet .}{MBC of EDTA alone}$$

Deviations from FICI or FBCI=1 indicate interactions: FICI>1 shows negative interaction, while FICI<1 indicates a positive one. Practical limits define synergy as FICI≤0.5 and antagonism as FICI≥4.

TABLE 3
MIC in Combination

| | MIC in Combination | | |
|---|---|---|---|
| | T-EDTA | Tetracycline | |
| Strains | (wt %) | (μg/mL) | FICI |
| E. Coli | 0.25 | 8 | 0.3 (Synergy) |
| HC1-R2A-1 | | | |
| S. epidermidis | 0.015 | 0.5 | 0.3 (Synergy) |
| HC3-CA-1 | | | |

TABLE 2
MBC in Combination

| | MBC in Combination | | |
|---|---|---|---|
| | T-EDTA | Tetracycline | |
| Strains | (wt %) | (μg/mL) | FBCI |
| E. Coli | 0.25 | 8 | 0.25 |
| HC1-R2A-1 | | | (Synergy) |
| S. epidermidis | 0.25 | 1 | 0.18 |
| HC3-CA-1 | | | (Synergy) |

The results clearly show a synergistic effect from the combination of the antimicrobial (tetracycline) and the EDTA by reducing the MIC and MBC of both the EDTA and the antimicrobial.

The term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower), preferably 15 percent, more preferably 10 percent, and most preferably 5 percent.

When introducing elements of aspects of the invention or aspects thereof, the articles “a,” “an,” “the,” and “said” are intended to mean that there are one or more of the elements. The terms “comprising,” “including,” and “having” are inclusive and mean that there may be additional elements other than the listed elements.

The term “therapeutically effective amount” means an amount of active ingredient(s) effective to prevent, slow, alleviate or ameliorate symptoms of a disorder or condition, or to prolong the survival of the subject being treated. Determination of a therapeutically effective amount of an ingredient is within the capability of those skilled in the art, especially in light of the detailed disclosure provided herein.

The term “pharmaceutical composition” is defined as a chemical or a biological compound or substance, or a mixture or combination of two or more such compounds or substances, intended for use in the medical diagnosis, cure, treatment, or prevention of disease or pathology. As used herein “pharmaceutical composition” and “medicament” may be used interchangeably.

The term “treat” or “treatment” refers to administration of an active pharmaceutical ingredient to a subject aimed at preventing, alleviating, and/or curing a medical condition, disease, or disorder.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing makes reference to particular illustrative embodiments, these examples should not be construed as limitations. The inventive system, methods, and products can be adapted for other uses or forms not explicitly listed above and can be modified in numerous ways within the spirit of the present disclosure. Thus, the present invention is not limited to the disclosed aspects, but is to be accorded the widest scope consistent with the claims below.

We claim:

1. A method for treating an infection in a patient in need thereof at reduced risk of creating disease-resistant organisms, comprising providing a therapeutically effective dose of an antimicrobial susceptible to drug-resistant organisms provided in an aqueous medium comprising a therapeutically effective amount of EDTA at a pH from 8.5 to 11, and delivering the aqueous medium via an aerosol delivery device to the patient.

2. The method of claim 1, wherein the infection is a respiratory infection and wherein the aqueous medium further comprises at least one of the group consisting of an NO-generating compound, N-acetyl cysteine, bromhexine, ectoin, and an enzyme inhibitor adapted to inhibit enzymes known to assist drug resistance relative to the antimicrobial.

3. The method of claim 1, wherein the infection is a respiratory infection and wherein the antimicrobial is selected from amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, and voriconazole.

4. The method of claim 1, wherein application of the aqueous medium is effective in reducing an infection of the lungs, sinuses, mouth, or throat.

5. The method of claim 1, wherein the infection is associated with cystic fibrosis, pneumonia, COPD, or bronchitis.

6. An aerosol delivery system for treating a respiratory ailment in a patient in need thereof, wherein the system comprises a nebulizer having a nebulizing chamber and a pharmaceutical composition comprising a therapeutically effective amount of one or more antimicrobials and EDTA at a pH of 8.5 to 11, wherein the pharmaceutical composition is nebulized in the nebulizing chamber and delivered as an aerosol to a patient.

7. The system of claim 6, wherein the pharmaceutical composition is nebulized by the action of an ultrasonically activated horn or mesh.

8. The system of claim 6, wherein the pharmaceutical composition is nebulized by the action of a pressurized gas.

9. The system of claim 6, wherein the pharmaceutical composition further comprises water, a buffer system, and wherein the active pharmaceutical ingredient comprises an aminoglycoside or a peptide.

10. A method employing the system of claim 6, wherein the system is used to treat a biofilm, and wherein the medicament further comprises a biofilm mitigation agent other than EDTA.

11. The system of claim 6, wherein the pharmaceutical composition comprises one or more salts selected from carbonates, phosphates, citrates and bicarbonates.

12. The system of claim 6, further comprising a monitoring unit to monitor one or more of the user's breathing behavior, the delivered dose of the pharmaceutical composition, the amount of pharmaceutical composition remaining, and the duration of inhalation of the aerosol.

13. A liquid pharmaceutical composition comprising:
an antimicrobial; and
EDTA, wherein the pharmaceutical composition has a pH of greater than 8.5.

14. The composition of claim 13, further comprising a mucolytic agent, a biofilm mitigation agent, an enzyme inhibitor, a NO-generating compound, or a combination thereof.

15. The composition of claim 13, further comprising a buffer, a solubilizer, a surfactant, a salt, an amino acid, a bronchodilator, a cough suppressant, or a combination thereof.

16. The composition of claim 13, wherein the antimicrobial comprises an antibiotic or an antifungal.

17. The composition of claim 16, wherein the antibiotic is selected from amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, and voriconazole.

18. The composition of claim 13, wherein the EDTA is present in a concentration from about 0.1 wt % to about 15 wt %.

19. The composition of claim 13, wherein the composition has a pH from 9 to 11.

20. The composition of claim 13, wherein the composition is suitable for administration to a patient via inhalation.

21. A method for treating an infection in a patient in need thereof, the method comprising administering to the patient a therapeutically effective amount of a composition of claim 13.

22. The method of claim 21, wherein the infection is caused by a bacteria.

23. A method for treating a biofilm in a patient in need thereof, the method comprising administering to the patient a therapeutically effective amount of a composition of claim 13.
